(19) United States
(12) Patent Application Publication
(10) Pub. No.: US 2008/0221382 A1
(43) Pub. Date: Sep. 11, 2008

(54) Method and system of radiotherapy enhancement through cellular perturbation using ultrasound and microbubbles

(75) Inventors: Raffi Karshafian, Scarborough (CA); Gregory Czarnota, Oakville (CA); Peter N. Burns, Toronto (CA)

Correspondence Address: Edward W. Bulchis, Esq.;DORSEY & WHITNEY LLP, Suite 3400, 1420 Fifth Avenue, Seattle, WA, 98101, US

(73) Assignee: Sunnybrook Health Sciences Centre, Toronto (CA)

(21) Appl. No.: 12/042,678

(22) Filed: Mar. 5, 2008

Related U.S. Application Data
(60) Provisional application No. 60/906,114, filed on Mar. 9, 2007.

(51) Int. Cl. A61N 5/00

(52) U.S. Cl. 600/2

(57) ABSTRACT

An embodiment of the invention is related to a system for providing radiotherapy to a treatment region. The system includes a radiation source and a sound source. The radiation source is positioned to irradiate the treatment region. The sound source is positioned to provide ultrasound to the treatment region so that the treatment region is subject to coincidental treatment by irradiation and ultrasound.

### CROSS-REFERENCE TO RELATED APPLICATION

This application claims the benefit of U.S. Provisional Application No. 60/906,114, filed Mar. 9, 2007.

### TECHNICAL FIELD

Embodiments of the invention relate to radiotherapy and, more particularly, to method and system of radiotherapy using ultrasound and microbubbles.

### BACKGROUND OF THE INVENTION

It has been found that microbubble agents exposed to ultrasound can perturb vascular endothelial cells in blood vessels, thereby rendering tissues and tumors more sensitive to the therapeutic effects of radiation. Microbubble contrast agents for ultrasound comprise microspheres of gas, usually air or a perfluorocarbon, stabilized by a thin shell of biocompatible material such as protein or lipid. A number of agents are approved for clinical use: one example is Definity made by Bristol-Myers Squibb of Boston Mass., which is perfluoropropane within a lipid shell. The median bubble diameter is 1-4 μm so that the bubbles can pass to the systemic circulation following peripheral venous injection. Microbubble contrast ultrasound imaging methods such as pulse inversion imaging exploit the nonlinear response of bubbles to an ultrasound field and allow real-time imaging of flowing or stationary bubbles in the vasculature, suppressing echoes from the tissue that surrounds them, and thus allowing perfusion imaging with ultrasound. These methods are widely available on clinical ultrasound scanners.

In addition to allowing visualization of the vasculature, acoustic exposure of bubbles at or near their resonant frequency can perturb the function of nearby cells; with effects including a reversible increase of cell membrane permeability. Phenomena related to acoustic bubble disruption, such as the formation of local microjets and shockwaves are capable of permeabilizing, as well as destroying a cell. Preliminary studies in laboratories have also shown the ability of ultrasound to enhance the uptake of drug analogues in a reversible manner that leaves the cell viable. Stable bubble oscillation and acoustic microstreaming are probably implicated, although there is little direct evidence. Proposed applications for this interaction include permeabilizing the blood-brain barrier for drug delivery, permeabilizing cells to introduce therapeutic agents or genes, and treating intravascular thrombi.

Bubbles can also be created in situ by the combined use of ultrasound and liquid droplets administered intravenously. For example, perfluorocarbon droplets can be vaporized by ultrasound to form gas bubbles. The advantage of such a method is that at the ultrasound exposures below the vaporization threshold the fluid droplets are practically transparent to the propagating ultrasound field and thus the cavitation or vaporization effect can be localized completely at the desired location.

In general, radiation is a major anti-cancer therapy and is currently used to treat a majority of patients with tumors in Europe and North America. Canonical radiobiology recognizes that radiation acts primarily by damaging cancer-cell DNA leading to cell death. However, emerging data indicate that radiation-induced apoptotic effects on blood-vessel cells can lead to vascular destruction and subsequent secondary tumor-cell death. In this view, it is the endothelial cell lining the vasculature that is proposed as the primary target for radiation. It is postulated that this vascular death may be an important mechanism of tumor kill in vivo, so that tumor cells die secondarily to damage caused by radiation to the microvasculature. Though the relative contributions of radiation-induced vascular effects and clonogenic tumor-cell death are not fully understood, efforts to optimize radiation treatment attempt to account for vascular effects, for example, by using drugs that target tumor blood vessels in combination with radiation.

It has been demonstrated that single large doses of radiation preferentially damage gut endothelial cells causing apoptosis and that epithelial stem-cell death is secondary to this. These results have been recapitulated in lung and brain tissue. Conversely, basic fibroblast growth factor (bFGF), a vascular protective agent, enhances epithelial stem-cell survival from whole-body irradiation. Some studies conclude that early phase microvascular endothelial apoptosis is mandatory for tumor cure. Other work has shown that tumors grown in apoptosis resistant mice, with deficiency in asmase (acid-sphingomyelinase) or bax (a pro-apoptotic member of the Bcl-2 family of proteins), were completely resistant to 15-20 Gy single dose irradiation. The same asmase-deficient mice, lacking a gene for acid-sphingomyelinase, an enzyme enriched in endothelial cells and required for apoptosis, were also protected from other radiation effects. Thus it is suggested that radiation-induced lesions in tumor cells were by themselves not lethal but their conversion to lethal damage is connected to the endothelial cell function.

These results lead to the idea of modifying tumor radiosensitivity by inhibiting endothelial-cell protectors and decreasing endothelial cell stimulators, thus increasing radiosensitivity with better treatment outcomes using lower doses of radiation. To date, pharmacological or chemical agents have been used. Among other problems, these methods of administration are not spatially specific in the body, and can create problems of toxicity: for example, histone-deacetylase inhibitors are effective radiosensitizers whose application is limited by high toxicity.

There is, therefore, a need for a method and system to improve effects of radiotherapy on tumorous tissue while minimizing effects on neighboring normal tissue.

### SUMMARY OF THE INVENTION

An embodiment of the invention is related to a system for providing radiotherapy to a treatment region. The system includes a radiation source and a sound source. The radiation source is positioned to irradiate the treatment region. The sound source is positioned to provide ultrasound to the treatment region so that the treatment region is subject to coincidental treatment by irradiation and ultrasound.

Another embodiment of the invention is related to a method of providing radiotherapy to a treatment region. According to the method, microbubbles are provided within vasculature of the treatment region. The treatment region is exposed to ultrasound to cause perturbation of vascular endothelial cells within the treatment region. The treatment region is also exposed to radiation.

### BRIEF DESCRIPTION OF THE DRAWINGS

FIG. 1 shows a system of coincident treatment of a target region by ultrasound and radiotherapy in accordance with an embodiment of the invention.

FIG. 2 shows administration of microbubbles externally into a target region by intravascular injection in accordance with an embodiment of the invention.

FIG. 3 shows bubble creation in a target region by externally applied ultrasound in accordance with an embodiment of the invention.

### DETAILED DESCRIPTION OF PREFERRED EMBODIMENTS

Certain details are set forth below to provide a sufficient understanding of embodiments of the invention. However, it will be clear to one skilled in the art that embodiments of the invention may be practiced without these particular details. Moreover, the particular embodiments of the present invention described herein are provided by way of example and should not be used to limit the scope of the invention to these particular embodiments.

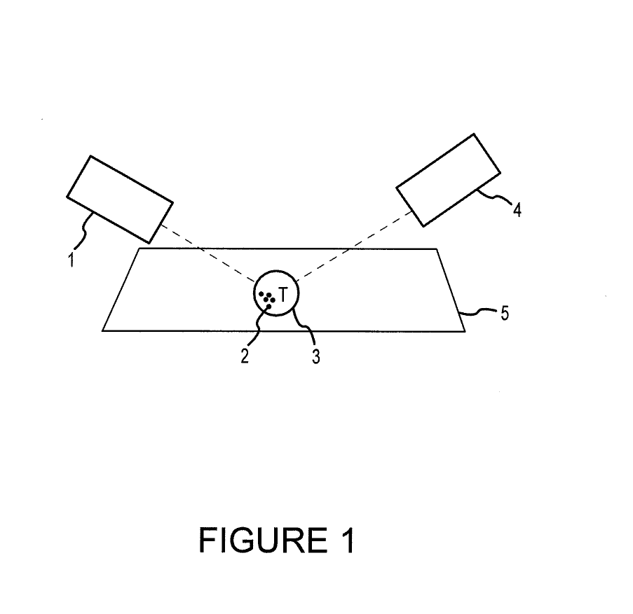

FIG. 1 shows a system of coincident treatment of a target region by ultrasound and radiotherapy in accordance with an embodiment of the invention. A target region T, such as a tumor, in a tissue 3 is irradiated with a radiation source for radiotherapy. The radiation source may be an external beam 1 or an internally placed source 2 within the target region T, as in brachytherapy. An ultrasound transducer 4 is positioned to irradiate the target region T with sound waves. As an alternative to using an external ultrasound transducer, such as the transducer 4, an internal ultrasound transducer (not shown), such as a transducer introduced through a catheter, may be used. During the sound radiation, microbubbles may be present within the target region T of tissue 3. An imaging system 5, such as ultrasound, computed tomography or magnetic resonance imaging, may be used to co-localize the ultrasound and radiotherapy treatments.

With the system depicted in FIG. 1, a subject with tumor, whether human or animal, may first be imaged with ultrasound or other imaging method to locate the tumor in preparation for treatment. Next, in one embodiment of the invention, the subject is given a bolus or a continuous infusion of microbubble-based intravenous contrast agent (targeted or non-targeted) and the tumor vasculature is monitored with the chosen imaging method until there is a desired or maximal microbubble concentration within the tumor vasculature. In an alternative embodiment of the invention, the subject is treated with ultrasound with or without the administration of an exogenous material, such as perfluorocarbon liquid droplets, so as to produce gas bodies within the treatment region until there is a desired or maximal microbubble concentration within the tumor vasculature.

When there is a desired or maximal microbubble concentration within the tumor vasculature, the subject may be exposed to ultrasound, directed at the tumor or target area so as to expose the tumor or target area with a predetermined set of parameters, such as mechanical index, frequency, pulse duration and repetition frequency for a preset period of time. This is intended to cause microbubble oscillation and/or disruption to result in perturbation of vascular endothelial cells within the tumor while minimizing effects of the treatment outside of the tumor. The above ultrasound parameters may be controlled through information derived from real time ultrasound imaging. Afterwards, the subject may undergo radiotherapy by being exposed to radiation. Alternatively, the administration of radiation may precede or be carried out simultaneously with the aforementioned procedures of imaging the subject to locate the tumor, administration of intravenous contrast agent or administration of an exogenous material to produce microbubbles in the treatment region, and exposure to ultrasound.

This process may be repeated with every radiation fraction during a course of treatment or with selected fractions. It may be used with fractionated or non-fractionated treatment. It may be used with external beam radiation, brachytherapy (intracavitary, interstitial or other), or targeted radiation treatments (for instance, but not limited to, radioconjugated antibodies). Embodiments of the invention may be used in conjunction with therapeutic agents, such as drugs that are targeted to disrupt or inhibit the vasculature. The ultrasound exposure may be guided by ultrasound imaging, other imaging methods known in the art or yet to be developed, or a combination of imaging methods.

An embodiment of the invention includes a device that provides ultrasound-mediated microbubble cellular perturbation which enhances the response of cells to radiation. Various embodiments of the invention include its use in vivo to enhance tumor responses to radiation by perturbing the vasculature and should permit radioenhancement to be conformally targeted to a tumor, thus minimizing effects on neighboring normal tissue. The method is used prior to, during, or shortly after the delivery of radiation to enhance and localize the therapeutic effects of radiation. Embodiments of the invention are applicable not only to elicit the conformal targeting of radioenhancement prior to or after external beam radiation, but can also be used to conformally target radioenhancement prior to or after brachytherapy or other modes of delivery of radiotherapy.

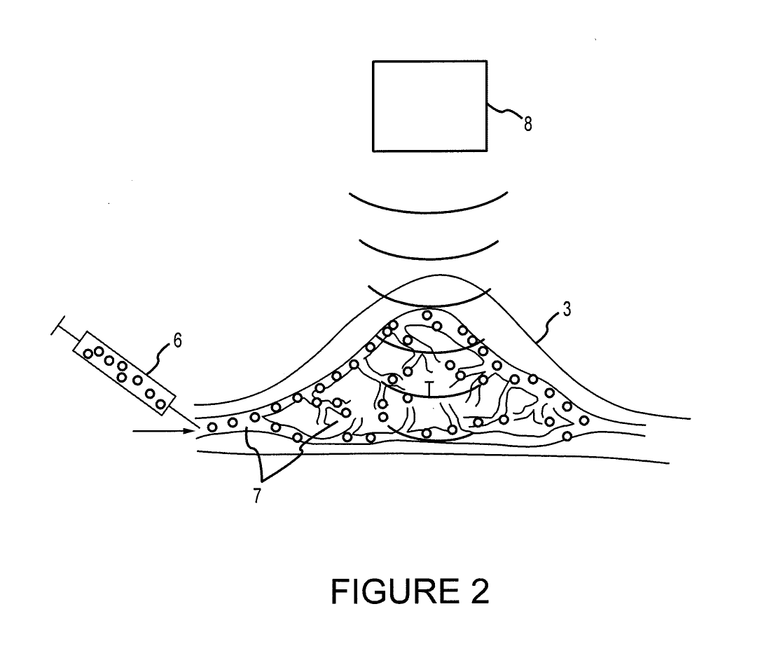

FIG. 2 shows administration of microbubbles externally into a target region by intravascular injection in accordance with an embodiment of the invention. In one embodiment, gas filled microbubbles, such as those used as contrast agents for diagnostic ultrasound, are administered by an intravascular injector 6 and carried to the target region T by blood flow 7. Alternatively, in accordance with an embodiment of the invention, microbubbles may be created in a target region of the tissue 3 solely by external activation, such as, for example, by an ultrasound transducer 8. The goal is to sensitize the target region T to radiotherapy by exposure to ultrasound in the presence of microbubbles, although the radiotherapy can alternatively be used before or after the microbubbles are exposed to ultrasound.

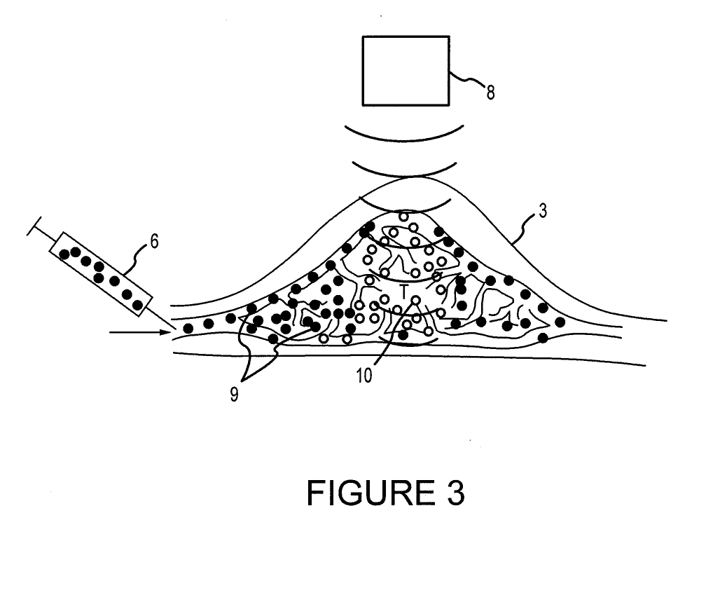

FIG. 3 shows the formation of microbubbles 10 in the vasculature from seeds 9, such as liquid droplets, injected from a syringe 6 into the vasculature. Once the seeds 9 have been injected and are in place in the vasculature and tissues, they are exposed to ultrasound from the transducer. The ultrasound causes the microbubbles 10 to be formed from the seeds by either cavitation or vaporization. Seeds for the production of microbubbles may comprise droplets of low-solubility liquids such as perfluorocarbons, and may be administered to the target region T to facilitate the production of microbubbles. Droplets used as microbubble seeds may be untargeted or they may be targeted with a ligand or other agent to a specific biological target so that the microbubbles will be concentrated in the biological target. The droplets may alternatively or additionally be loaded with drugs such as radiosensitizer drugs that enhance the radiotherapy. Such drugs are often very toxic to tissues, but they are not released until the microbubbles 10 are created from the droplets in the tumor. As a result, there is relatively slight damage to other tissues. Another advantage to using droplets to create the microbubbles 10 is the droplets can be made smaller than microbubbles, and they are therefore able to leave the vasculature and diffuse into tissues.

Embodiments of the invention may include the above process as well as any technology that enables the process or is used in connection with the process or to carry it out. One implementation is by using a stand-alone ultrasound unit or an ultrasound device combined with a radiotherapy device whereby the ultrasound device is used to visualize the tumor. This or a separate ultrasound device may also be used to selectively perturb the vascular endothelial cells within the tumor so as to conformally radiosensitize the tumor.

As will be appreciated by those ordinarily skilled in the art, embodiments of the invention provide a technique to improve effects of radiotherapy on tumorous tissue while minimizing effects on neighboring normal tissue by use of ultrasound and microbubbles. The effect of the ultrasound treatment is to sensitize the target region T to the effects of radiotherapy. The disclosed procedure relies on the fact that microbubble agents exposed to ultrasound can perturb vascular endothelial cells in blood vessels, thereby rendering tissues and tumors more sensitive to the therapeutic effects of radiation. The radiotherapy might be administered by irradiation from an external beam or by the implantation of radioactive sources in the target region, such as in brachytherapy. The radiotherapy may be administered before, during or after the ultrasound treatment. An imaging system can be used to co-localize the ultrasound and radiotherapy treatments.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A system for providing radiotherapy to a treatment region, comprising:
a radiation source positioned to irradiate the treatment region; and
a sound source positioned to provide ultrasound to the treatment region so that the treatment region is subject to coincidental treatment by irradiation and ultrasound.

2. The system of claim 1, further comprising an imaging unit operable to locate the treatment region so that irradiation and ultrasound can be localized to the treatment region for coincidental treatment.

3. The system of claim 1 wherein the radiation source is an external beam radiation device.

4. The system of claim 1 wherein the radiation source is a radioactive material used in brachytherapy.

5. The system of claim 1, further comprising at least a device for providing microbubbles within vasculature of the treatment region at least during a period of time when the ultrasound is provided to the treatment region.

6. The system of claim 5 wherein the microbubbles are administered externally by intravascular injection.

7. The system of claim 5 wherein the microbubbles are created by external activation.

8. The system of claim 5 wherein the microbubbles are created by an ultrasound transducer.

9. The system of claim 7 wherein the microbubbles are formed from seeds within the treatment region or vessels of the treatment region.

10. A method of providing radiotherapy to a treatment region, comprising:
providing microbubbles within vasculature of the treatment region;
exposing the treatment region to ultrasound to cause perturbation of vascular endothelial cells within the treatment region; and
exposing the treatment region to radiation.

11. The method of claim 10, further comprising locating the treatment region in the treatment region by imaging with ultrasound.

12. The method of claim 10 wherein providing microbubbles within vasculature of the treatment region comprises providing microbubble-based intravenous contrast agent to produce microbubbles within vasculature of the treatment region.

13. The method of claim 10 wherein providing microbubbles within vasculature of the treatment region comprises producing microbubbles within vasculature of the treatment region using ultrasound.

14. The method of claim 13 wherein producing microbubbles within vasculature of the treatment region using ultrasound comprises producing microbubbles within vasculature of the treatment region using ultrasound and having an exogenous material administered into the treatment region.

15. The method of claim 14 wherein the exogenous material comprises perfluorocarbon liquid droplets.

16. The method of claim 10 wherein exposing the treatment region to ultrasound comprises exposing the treatment region to ultrasound having at least one of mechanical index, frequency, pulse duration and repetition frequency for a preset duration at a respective predetermined value.

17. The method of claim 10 wherein exposing the treatment region to radiation comprises exposing the treatment region to an external beam radiation.

18. The method of claim 10 wherein exposing the treatment region to radiation comprises providing brachytherapy to the treatment region.

19. The method of claim 10 wherein exposing the treatment region to radiation comprises providing targeted radiation treatment to the treatment region.

20. The method of claim 10 wherein the act of exposing the treatment region to radiation is performed before the act of exposing the treatment region to ultrasound.

21. The method of claim 10 wherein the acts of exposing the treatment region to radiation and exposing the treatment region to ultrasound are performed simultaneously.

22. The method of claim 10 wherein exposing the treatment region to radiation comprises exposing the treatment region to radiation in fractions, and wherein the act of exposing the treatment region to radiation is repeated with every fraction of radiation.

23. The method of claim 10 wherein exposing the treatment region to radiation comprises exposing the treatment region to radiation in fractions, and wherein the act of exposing the treatment region to ultrasound is repeated with selected ones of the fractions of radiation.

24. A method of providing radiotherapy to a treatment region, comprising:
providing irradiation to the treatment region;
providing ultrasound to the treatment region; and
localizing the irradiation and ultrasound to the treatment region to allow coincident treatment of the treatment region by sound and radiotherapy.

25. The method of claim 24 wherein localizing the irradiation and ultrasound comprises localizing the irradiation and ultrasound using an imaging device.
